(19) United States
(12) Patent Application Publication
(10) Pub. No.: US 2022/0313470 A1
(43) Pub. Date: Oct. 6, 2022

(54) PATIENT IMMOBILIZATION

(71) Applicant: Children's Medical Center Corporation, Boston, MA (US)

(72) Inventors: Kristen Cuzmanes, Austin, TX (US); Michele Linda DeGrazia, Lakeville, MA (US); Jennifer Haley, Marshfield, MA (US)

(21) Appl. No.: 17/711,256

(22) Filed: Apr. 1, 2022

Related U.S. Application Data
(60) Provisional application No. 63/184,977, filed on May 6, 2021.
(60) Provisional application No. 63/170,606, filed on Apr. 5, 2021.

(51) Int. Cl. A61F 5/37

(52) U.S. Cl. CPC A61F 5/3723

(57) ABSTRACT

A wrap for a post-catheterization patient (or the like) may include an apparatus that covers the patient's torso for warmth and comfort, while generally immobilizing the patient's legs and lower body in a supine position with their legs slightly separated and immobilized laterally to discourage bending of the legs and/or rotation of the patient. The device may allow for relatively unrestricted movement of a patient's shoulders, head, and arms, thus providing a feel of mobility and permitting the patient (e.g., a toddler) to interact with their surroundings. Moreover, when a patient is secured in the wrap, treatment areas such as the groin (e.g., for catheter placement) and lower extremities may be exposed for examination and treatment by a caregiver.

### CROSS-REFERENCE TO RELATED APPLICATIONS

This application claims priority to U.S. Provisional Patent App. No. 63/184,977 filed on May 6, 2021, and U.S. Provisional Patent App. No. 63/170,606 filed on Apr. 5, 2021, where the entire contents of each of the foregoing applications are hereby incorporated by reference.

TECHNICAL FIELD

The present disclosure relates to devices, systems, kits, and methods for patient immobilization, such as an apparatus featuring a wrap that stabilizes a patient (e.g., a post cardiac catheterization pediatric patient) while also providing access for practitioners (e.g., to a surgical site and/or areas for health monitoring) and comfort for the patient (e.g., allowing a patient to move/use their arms).

### BACKGROUND

Most cardiac catheterizations in young pediatric patients are performed via the femoral arterial and venous access sites, and require 4-6 hours of immobilization and supine bed rest following the procedure to maintain hemostasis and prevent the occurrence of re-bleeding. Current bleeding prevention practices for these patients are highly variable and are not standardized across the medical community. For example, a bath blanket may be used to swaddle a pediatric patient's lower body for immobilization; and, while attempts are made by nurses to leave a window to visualize the catheter insertion site, it is not always possible. Thus, this technique often requires the unwrapping and re-wrapping of a patient to check access sites for re-bleeding, and the bath blanket itself has a tendency to come loose and to open, even from relatively limited patient movement depending upon the patient's size and strength. Currently, there is a lack of devices and techniques that can help manage re-bleeding risks in post-cardiac catheterization patients from 1-5 years of age. Additionally, there is a lack of devices that can enable a parent or caregiver to play an active role in a pediatric patient's recovery, such as by holding the patient.

There remains a need for improved techniques for stabilizing and providing comfort to a patient after a procedure, such as a young pediatric patient following a cardiac catheterization procedure.

### SUMMARY

A wrap for a post-catheterization patient (or the like) may include an apparatus that covers the patient's torso for warmth and comfort, while generally immobilizing the patient's legs and lower body in a supine position with their legs slightly separated and immobilized laterally to discourage bending of the legs and/or rotation of the patient. The device may allow for relatively unrestricted movement of a patient's shoulders, head, and arms, thus providing a feel of mobility and permitting the patient (e.g., a toddler) to interact with their surroundings. Moreover, when a patient is secured in the wrap, treatment areas such as the groin (e.g., for catheter placement) and lower extremities may be exposed for examination and treatment by a caregiver.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a wrap for patient immobilization.

FIG. 2 shows a kit for patient immobilization.

FIG. 3 shows a rear view of an apparatus for patient immobilization.

FIG. 4 shows an apparatus for patient immobilization positioned for use with a patient.

FIG. 5 shows a stabilizer being positioned for use within an apparatus for patient immobilization.

FIG. 6 shows an immobilized patient.

FIG. 7 is a diagram of a wrap.

FIG. 8 is a flow chart of a method for patient immobilization.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “above,” “below,” and the like, are words of convenience and are not to be construed as limiting terms.

The present teachings may include devices, systems, kits, and methods featuring a wrap for a patient that provides desired immobilization and/or stabilization for the patient without overly restraining the patient. The wrap may further include one or more windows or other viewing areas, e.g., so that medical personnel can view and/or access a surgical site or other area of interest on the patient's body. Moreover, the wrap may leave the lower extremities of the patient exposed (e.g., a patient's feet), such that medical personnel can access such extremities for examination and the like. The present teachings may be particularly advantageous for pediatric patients such as toddlers or other small children, where such patients may have issues staying immobile as recommended or prescribed after a procedure, although other patients could also or instead benefit from the device (e.g., patients with learning or developmental disabilities, and the like). In particular, the present teachings may be advantageous for pediatric post cardiac catheterization patients.

Thus, an aspect of the present teachings includes a post cardiac catheterization wrap apparatus that is designed to promote and assist with lower body immobilization and/or stabilization. More specifically, the apparatus may be structurally configured to promote immobility and prevent complications in young pediatric patients following a cardiac catheterization procedure. The apparatus may be adjustable to accommodate children 0-5 years of age and of varying sizes (weight and length). The apparatus may be structurally configured to minimize the risk of re-bleeding, to increase visualization of one or more areas of interest (e.g., a groin access site from a catheterization procedure), and/or to promote provider/patient interaction, thereby aiding in decreasing a patient's anxiety following a procedure such as a cardiac catheterization procedure. Thus, the apparatus may enable a parent or caregiver to play an active role in a pediatric patient's recovery (e.g., potentially holding the patient), thereby providing a focus on family-centered care and the like.

An aspect of the present teachings therefore includes a medical safety device that swaddles a patient in a supine position while helping to keep the patient from bending at their waist. The device may allow a provider to visualize/palpate groin access sites without un-wrapping the patient, thereby potentially decreasing patient agitation. The device thus may have the potential to offer protection from complications of re-bleeding.

Although primarily described for use with pediatric post cardiac catheterization patients, devices and techniques described herein may have other applications as will be understood by a skilled artisan. For example, the present teachings may be used for patients undergoing neurological embolization procedures that require supine bedrest, or similar.

It will be understood that, as used herein, “immobilization” (or the like) of a patient includes a desired state for a patient, e.g., following a procedure or the like. In most aspects, this immobilization includes mitigating (or outright preventing) one or more of a bending at the waist of the patient, a bending of a knee of the patient, and rolling over (e.g., from a supine position on their back to a side position or a prone position, or vice versa). Also or instead, immobilization of a patient as described herein may include mitigating or preventing any movement of a patient's legs, with a possible exception of allowing for movement of a patient's feet (e.g., bending at the ankles or the like). However, it will be understood that “immobilization” (or the like) as used herein generally does not include a fully immobilized state. That is, in most implementations as described herein, and in preferred embodiments, the head of a patient and/or the arms of the patient are not substantially immobilized (contrary to the immobilization of the patient's legs, for instance). And, as described herein, this may be particularly advantageous for providing comfort to the patient, especially when the patient is relatively young or otherwise unaware, uncooperative, or the like.

The present description may use terms such as “device,” “apparatus,” “kit,” and/or “system” when describing aspects of the present teachings. In general, and unless expressly stated to the contrary, it will be understood that a “device” as used herein shall generally include an embodiment having a wrap as described herein; it will be understood that an “apparatus” as used herein shall generally include an embodiment featuring a wrap and a stabilizer as described herein; and it will be understood that a “kit” or “system” as used herein shall generally include an embodiment featuring a wrap and plurality of stabilizers as described herein.

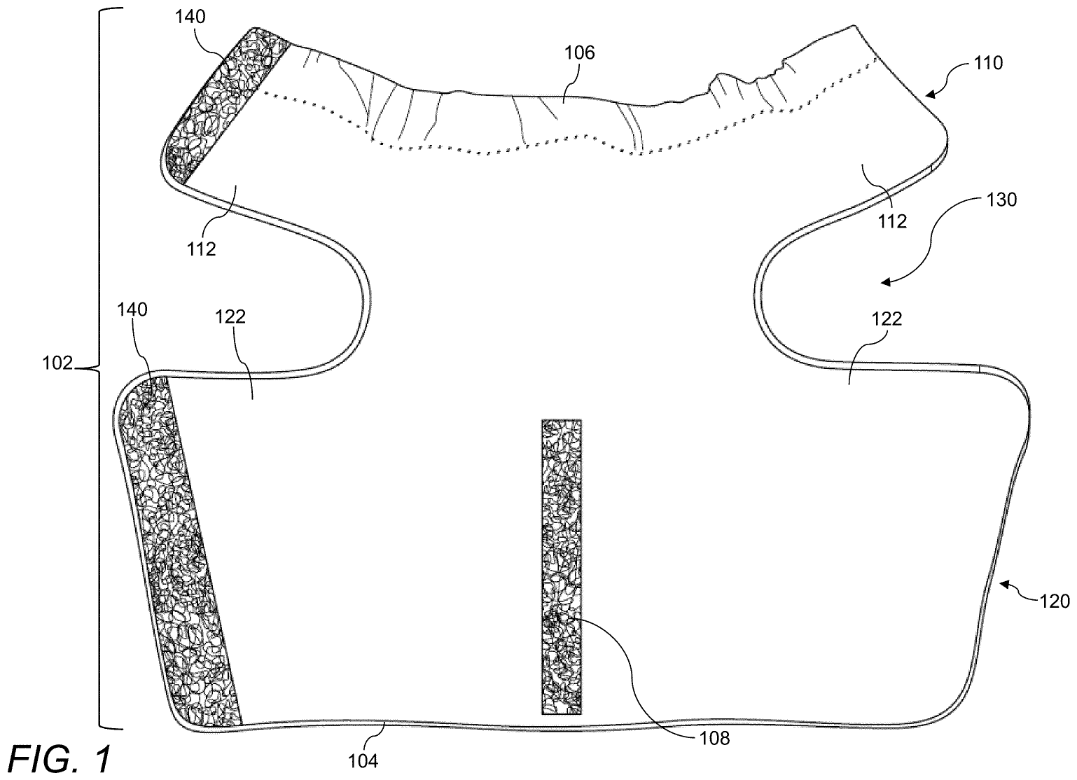

FIG. 1 shows a wrap for patient immobilization. The wrap 102 may form part of an overall apparatus for patient immobilization as described herein, and/or a portion of a kit or system for patient immobilization as described herein. The wrap 102 may be sized and shaped to enclose a patient in a supine position (and/or a prone position or another position) from below a pair of shoulders to above a pair of feet. In this manner, when the wrap 102 is placed for use about the patient, a pair of arms of the patient may be free to move while a pair of legs of the patient may be contained within the wrap. Thus, the wrap 102 may be structurally configured to at least partially swaddle a patient such that a substantial portion of a lower half (beneath the torso of the patient) is immobilized, while a portion of the upper half of the patient (above the torso of the patient) is free to move, such as the arms and head of the patient being free to move while the legs of the patient are substantially immobilized.

The wrap 102 may include a first panel 110 and a second panel 120 having one or more cutouts 130 at least partially disposed therebetween. That is, a cutout 130 may be formed in the wrap 102 such that the cutout 130 (and thus overall shape of the wrap 102) defines the first panel 110 and the second panel 120 of the wrap 102. As described herein, the cutouts 130 may be sized and shaped to form an opening within the wrap 102 that is positioned axially along the patient between the first panel 110 and the second panel 120 to provide a viewing region about a portion of interest for the patient (e.g., the groin region of the patient) when the wrap 102 is placed for use about the patient, e.g., when the patient is swaddled by the wrap 102.

Each of the first panel 110 and the second panel 120 may include flaps that are formed by the cutouts 130. That is, the first panel 110 may include a first pair of flaps 112 and the second panel 120 may include a second pair of flaps 122. The first pair of flaps 112 may be sized and shaped (and/or otherwise structurally configured) to at least partially surround a torso of the patient, while the second pair of flaps 122 may be sized and shaped (and/or otherwise structurally configured) to at least partially surround a pair of legs of the patient. And, more particularly, the second pair of flaps 122 may be sized and shaped (and/or otherwise structurally configured) to immobilize a lower portion of a patient's body, such as by preventing certain movement of legs of the patient, preventing bending of knees of the patient, preventing bending at the waist of the patient, and the like. In some aspects, the first pair of flaps 112 may be also or instead be sized and shaped (and/or otherwise structurally configured) to immobilize a portion of a patient's body, such as by preventing bending at the waist of the patient or the like.

One or more of the first panel 110 and the second panel 120 may include one or more couplers 140 thereon, e.g., coupled to or integral therewith. For example, in certain implementations, the first pair of flaps 112 includes a first coupler for releasably engaging the first pair of flaps 112 to one another about the torso of the patient; and similarly, in certain implementations, the second pair of flaps 122 may include a second coupler for releasably engaging the second pair of flaps 122 to one another about the legs of the patient. It will be understood that, using this example, the first coupler and the second coupler (of the one or more couplers 140 for the wrap 102 or apparatus, kit, or system in which the wrap 102 is used) may be the same type of coupler 140 or a different type of coupler 140.

By way of example, one or more of the couplers 140 (e.g., at least one of the first coupler and the second coupler) may include a hook and loop fastener. And, in certain aspects, such a hook and loop fastener (or another type of coupler 140) may extend across an entire width of at least one flap of the first pair of flaps 112 and the second pair of flaps 122 as shown in the figure. That is, an end of a flap of each of the first pair of flaps 112 and the second pair of flaps 122 may include a strip of a hook portion of a hook and loop fastener, which may be releasably couplable to a surface of an opposing flap (e.g., a back surface, such as the entire back surface of a flap or a substantially portion thereof), which may feature a loop portion of a hook and loop fastener (and/or a material structurally configured for engagement with the aforementioned hook portion or the like). In this manner, relatively wide sections of hook and loop fasteners (or the like) may be used, which can provide advantageous adherence for various sized patients. Also or instead, strips of hook and loop fasteners (or the like) may be used.

Other couplers 140 are also or instead possible, and thus it will be understood that a device according to the present teachings may include one or more attachment mechanisms and/or attachment areas. Other types of couplers 140 that may also or instead be used in the present teachings for engaging the flaps of one or more of the first pair of flaps 112 and the second pair of flaps 122 may include: projections and corresponding voids, bolts, clamps, clips, latches, pins, screws, sliders, snaps, buttons, combinations thereof, and the like.

In certain implementations, the wrap 102 may include an engagement area 108 structurally configured for receiving a stabilizer therein or thereon, such as any of the stabilizers described herein. In some aspects, this may include a strip of a hook and loop fastener and/or any of the other coupling elements described herein or known in the art. For example, in some aspects, a stabilizer as described herein may include a cooperating engagement region (e.g., a stabilizer may include its own cooperating hook and loop fastener or the like) that is structurally configured for coupling with the engagement area 108 of the wrap 102, e.g., releasably coupling with the engagement area 108 of the wrap 102. However, such a hook and loop fastener could pose a problem such as skin irritation, abrasions, and/or a pressure injury to legs of a patient, e.g., when a stabilizer is absent from the wrap 102 leaving this engagement area 108 exposed. Thus, in some aspects, the material used may be structurally configured to be relatively soft in order to avoid such abrasions, and/or the engagement area 108 may instead include another type of fastener. For example, in some aspects, the engagement area 108 may include a pocket or the like that is structurally configured to receive a portion of a stabilizer therein for alignment and/or securement to the wrap 102. Further to this example, a stabilizer may include an arm extending therefrom that can be positioned laterally within such a pocket of the engagement area 108—e.g., it can be slid into place within the pocket. Other configurations are also or instead possible. In other aspects, a stabilizer as described herein is permanently affixed to the wrap 102, e.g., via the engagement area 108 or otherwise. And, in yet some other aspects, there may be no affixing of the stabilizer to the wrap 102.

The wrap 102 may include a cotton material, although other materials are also or instead possible. For example, in an aspect, the wrap 102 may be made of a durable cotton material or the like. The wrap 102 may also or instead be made from a substantially waterproof material. The wrap 102 may include a binding trim 104 disposed at least partially about a perimeter of the wrap 102, which can provide for finished edging and enhance comfort for a patient immobilized by the wrap 102.

The wrap 102 may be made with a multilayer design (e.g., a triple layer design), e.g., having a quilted outer layer to promote stability and comfort. That is, in some aspects, the wrap 102 is formed of a plurality of layers. For example, the wrap 102 may be formed of at least three layers including an inner layer, a middle layer, and an outer layer. One or more of the plurality of layers of the wrap 102 may include a breathable material, or the wrap 102 may otherwise be at least partially fabricated from breathable materials. In an aspect, an inner layer of the wrap 102 is structurally configured to provide comfort to a patient (e.g., an inner lining of the wrap 102 may be made of a fabric that provides a relatively soft feel against a patient's skin, but that can also maintain relatively consistent temperatures for the patient), a middle layer of the wrap includes cushioning or the like for further comfort, and an outer layer may be structurally configured to provide one or more of stability, durability, sterility, and/or to promote engagement of a coupler 140. Other configurations are also or instead possible.

The wrap 102 (e.g., an inner lining of the wrap 102) may advantageously be formed of a breathable fabric or the like to facilitate air circulation, improve comfort, prevent hyperthermia, and/or the like. In a similar manner, a layer of the wrap 102 (e.g., a middle layer) may include foam cushioning or the like, which may advantageously be perforated to similarly facilitate air circulation, or the cushioning or the like may be formed of an open-cell foam material or other breathable material that permits a passage of air while also providing cushioning and/or support.

The wrap 102 may include one or more elastic sections 106 configured to conform to a shape of a patient. For example, and as shown in the figure, the wrap 102 may include an elastic section 106 disposed along a torso area for a patient. The size and shape of one or more of the elastic sections 106 may be configured to promote comfort and adherence for multiple size closures.

The wrap 102 may be sized and shaped for coverage of patients of variable sizes, e.g., the wrap 102 may have a width to accommodate variously-sized patients. In certain implementations, the wrap 102 is sized and shaped to immobilize patients between zero and five years of age.

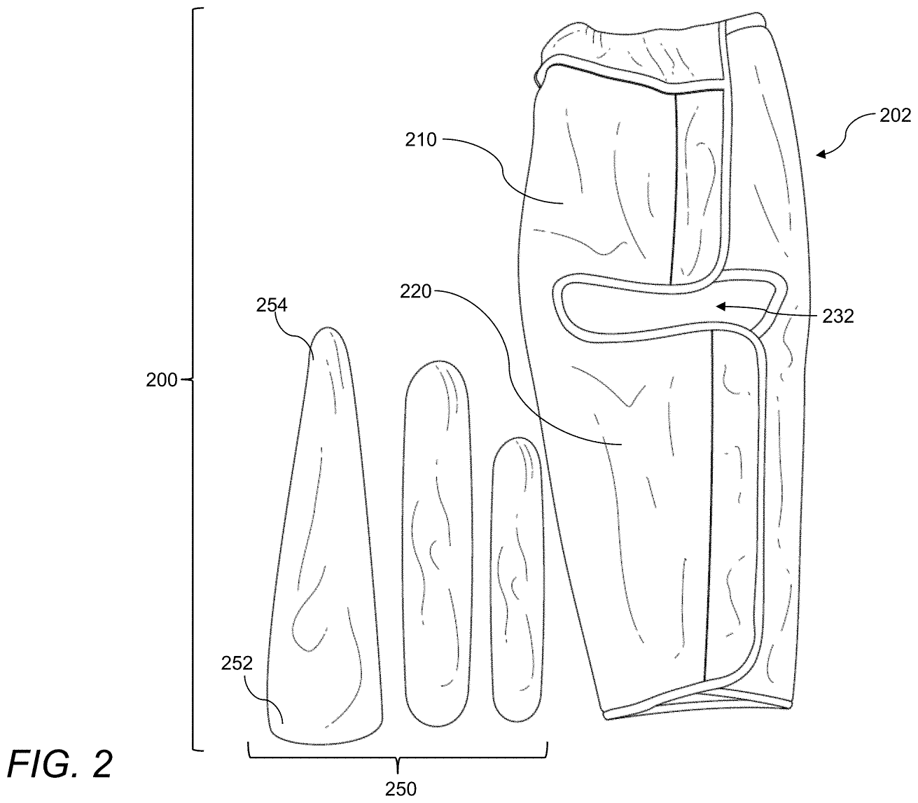

FIG. 2 shows a kit for patient immobilization. The kit 200 may generally include a wrap 202 and a plurality of stabilizers 250. It will be understood that the wrap 202 may be the same or similar to the wrap 102 described above with reference to FIG. 1.

Turning back to FIG. 2, the wrap 202 is shown with flaps of the first panel 210 engaged with one another and the flaps of the second panel 220 engaged with one another. In this manner, this figure shows how cutouts formed between the first panel 210 and the second panel 220 may form an opening 232 when the flaps of the panels are engaged with one another, and more particularly, when the first pair of flaps of the first panel 210 are engaged to one another about the torso of a patient and when the second pair of flaps of the second panel 220 are engaged to one another about a pair of legs of the patient. As shown in the figure, this opening 232 within the wrap 202 may be positioned axially across the wrap 202, e.g., along a mid-region of the wrap 202. This mid-region of the wrap 202 in which the opening 232 is formed may coincide with a region of interest for a patient immobilized by the wrap 202. For example, the region of interest may include a treatment region, such as the groin of the patient.

As stated above, the kit 200 may include a plurality of stabilizers 250. For example, and as shown in the figure, the kit 200 may include a plurality of stabilizers 250 where at least two of the plurality of stabilizers 250 have a different size and/or shape. In general, a stabilizer 250 may be structurally configured for positioning between legs of a patient to be immobilized using the wrap 202. In certain implementations, when positioned between a patient's legs, the stabilizer 250 may displace the pair of legs from one another to impart rotational stability to the patient when immobilized within the wrap 202. The stabilizer 250 may also or instead provide axial rigidity, e.g., axial rigidity sufficient to resist bending at a pair of knees of the patient when immobilized within the wrap 202.

One or more of the stabilizers 250 may be tapered. For example, a bottom end 252 of a stabilizer 250 may be laterally wider than a top end 254 of the stabilizer 250. In this manner, the bottom end 252 of such a stabilizer 250 may be configured for positioning substantially adjacent to ankles or feet of the patient, where the bottom end 252 acts to separate the patient's feet from one another. This may provide comfort and/or stability for the patient being immobilized by the wrap 202.

In some aspects, the stabilizer 250 may include a relatively soft material thereon or therein. In this manner, the stabilizer 250 may act as a cushion or the like between legs of the patient being immobilized by the wrap 202. For example, the stabilizer 250 may include a foam material or the like.

The stabilizer 250 may be engaged with the wrap 202. For example, the stabilizer 250 may be releasably couplable with the wrap 202, e.g., via a coupler such as a hook and loop coupler or the like. In this manner, the stabilizer 250 may be removable and replaceable relative to the wrap 202. In some aspects, the stabilizer 250 may be stitched onto an inner lining of the wrap 202, or otherwise substantially permanently attached to the wrap 202. In other aspects, the stabilizer 250 is an independent element that can be placed for use between a patient's legs (or otherwise) and/or removed without ever coupling to the wrap 202. In other aspects, the wrap 202 includes a pocket or the like for receiving a stabilizer 250 at least partially therein.

Thus, as described herein, devices, systems, kits, and methods according to the present teachings may include a stabilizer 250 formed as an intervening cushion that, inter alia, acts as a leg separator for a patient. The stabilizer 250 may be removable from the device (e.g., separable from the wrap 202). For example, the stabilizer 250 may include a removable foam leg separator, although other materials are also or instead possible. The stabilizer 250 may be one of a plurality of cushions having different sizes (e.g., lengths and/or widths) and/or shapes to facilitate comfort and prevent pressure-related injuries resulting from a patient's legs rubbing together, or otherwise. Thus, techniques as described herein may be part of a kit or system that includes one or more stabilizers 250. The stabilizer 250 may also or instead be stitched into a posterior wall of the inner lining of the wrap 202 to decrease skin irritation from a patient's legs rubbing together.

As discussed above, a stabilizer 250 for separating legs of the patient may advantageously be tapered so that the stabilizer 250 is laterally wider near the feet than it is near the groin. This can advantageously permit the stabilizer 250 to provide a greater separation at/near the ankles. In one aspect, this provides improved access to the feet for assessment of peripheral perfusion. In another aspect, this provides lateral stabilization and promotes rotational stability, while still traveling along and thus supporting/restraining substantially all of the interior surfaces of the adjacent legs. Additionally, the stabilizer 250 can be made in multiple sizes to accommodate different use cases, such as by adapting to different sized children or providing different degrees of ankle separation.

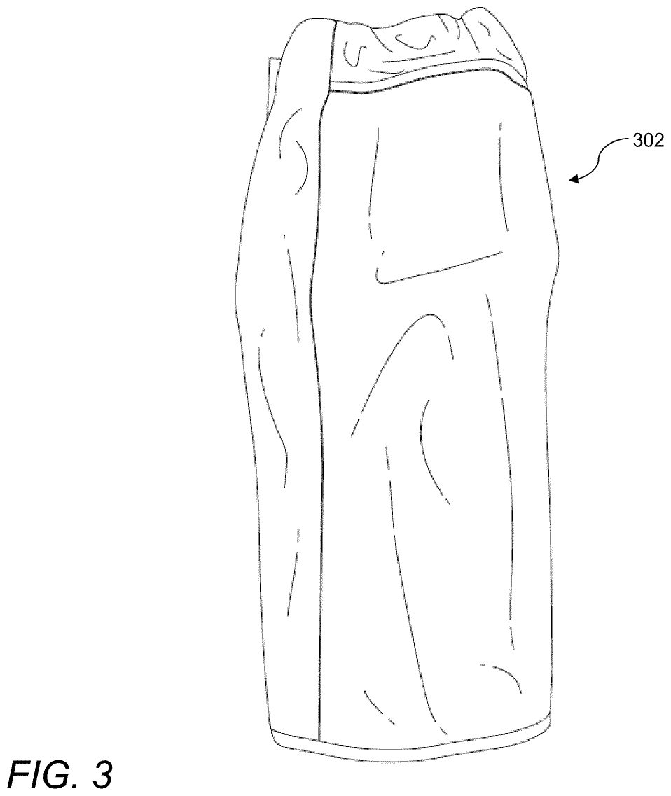

FIG. 3 shows a rear view of an apparatus for patient immobilization. As discussed herein, the apparatus may include a wrap 302, which may be the same or similar to any as described herein.

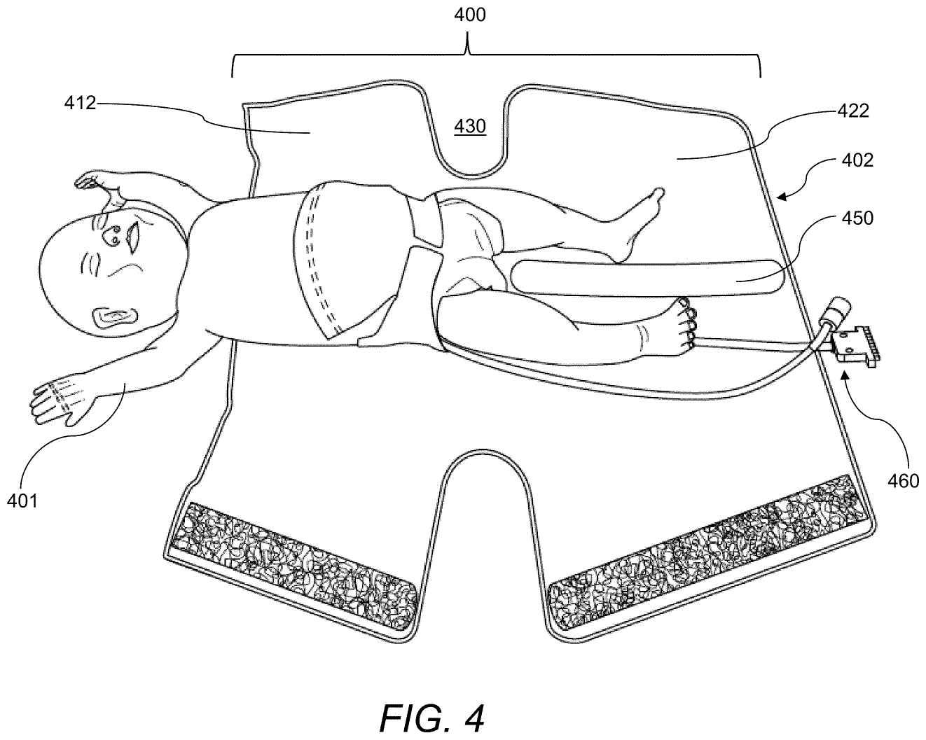

FIG. 4 shows an apparatus for patient immobilization positioned for use with a patient. Specifically, this figure shows an apparatus 400 including a wrap 402 and a stabilizer 450, which may be the same or similar to any of those as described herein.

The patient 401 may be positioned with the wrap 402 beneath them while in a supine position. More particularly, the wrap 402 may be placed beneath the patient 401, who is disposed in a supine position, with a pair of shoulders of the patient 401 positioned above the wrap 402. Although not shown in this figure, a pair of feet of the patient 401 may be positioned below the wrap 402 so that feet of the patient 401 will remain unswaddled from the wrap 402 when in use. In other aspects, as shown in the figure, the feet of the patient 401 may not extend beyond the wrap 402, but feet of the patient 401 may still be accessible from a bottom end of the wrap 402 that remains open when the patient 401 is swaddled by the wrap 402. Regardless, the patient 401 may be positioned in a manner such that a first pair of flaps 412 of a first panel of the wrap 402 can be engaged about the torso of the patient 401, and such that a second pair of flaps 422 of a second panel of the wrap 402 can be engaged about the legs of the patient 401. Also, or instead, the patient 401 may be positioned relative to the wrap 402 such that a cutout 430 formed between panels of the wrap 402 is disposed substantially adjacent to a region of interest for the patient 401, such as a groin area for treatment or other care, or the like.

In addition to showing the patient 401 positioned for swaddling by the wrap 402, the figure also shows the stabilizer 450 positioned between a pair of legs of the patient 401. In this manner, the stabilizer 450 may act to displace the pair of legs from one another to impart rotational stability to the patient 401 when immobilized within the wrap 402. The stabilizer 450 may also or instead provide axial rigidity to resist bending at a pair of knees of the patient 401 when immobilized within the wrap 402.

The apparatus 400 may accommodate medical equipment 460 in addition to the patient 401. This may include one or more of cabling, tubing (e.g., intravenous (IV) tubing), wires, and the like. This may also or instead include one or more of medicines (e.g., IV medications or otherwise), bandages, diapers, clothing, and the like. The medical equipment 460 may also or instead include one or more of surgical instruments (or other instruments), prosthetics, catheters, and the like. By way of example and as shown in the figure, one or more cables and/or wires may be placed for use with the patient 401 within the wrap 402, where ends of such medical equipment 460 can be accessible via one or more openings in the wrap 402.

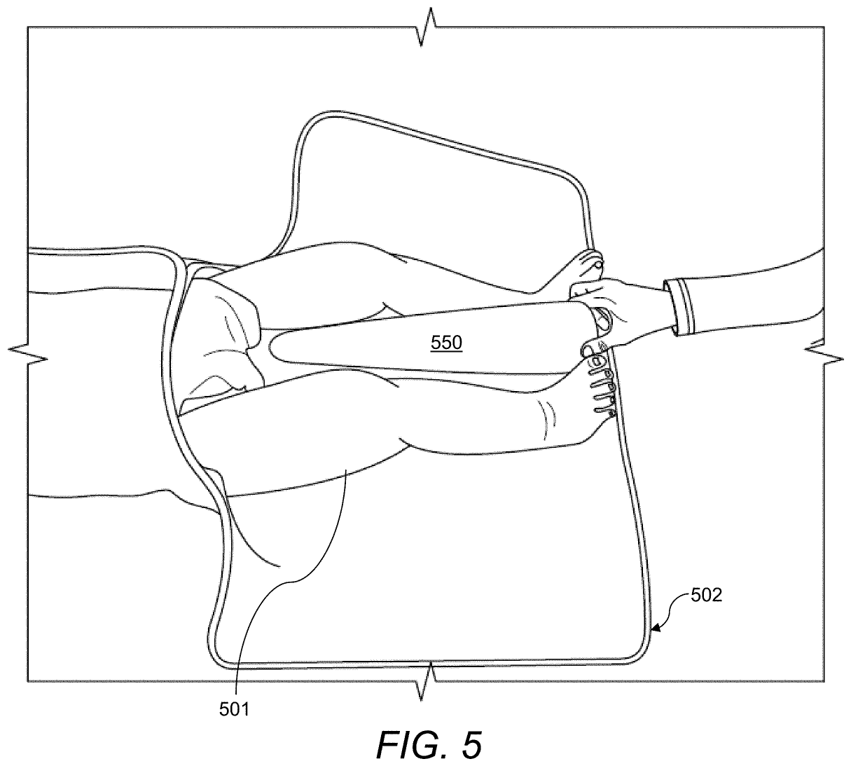

FIG. 5 shows a stabilizer being positioned for use within an apparatus for patient immobilization. In particular, the stabilizer 550 is shown being manually placed within the wrap 502 between a pair of legs of the patient 501 to displace the legs from one another to impart rotational stability to the patient 501 when immobilized within the wrap 502.

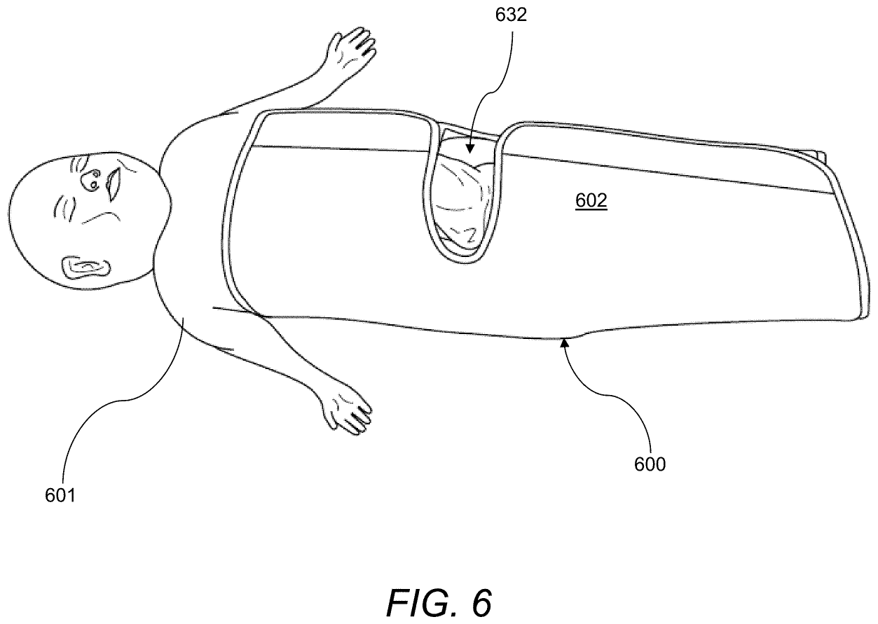

FIG. 6 shows an immobilized patient. As shown in the figure, using an apparatus 600 according to the present teachings, a patient 601 may be at least partially immobilized with their arms and head free to move in a predetermined manner, but with their legs immobilized. Further, an opening 632 may be defined by the apparatus 600 while in use immobilizing a patient 601, where the opening 632 is positioned axially along the patient 601 to provide a viewing region about a region of interest—e.g., a groin of the patient 601. In some aspects, the viewing region formed by the opening 632 may be exposed to provide access to at least a portion of the patient 601 through the viewing region when the patient 601 is immobilized within the wrap 602. In other aspects, the opening 632 may be at least partially covered.

Thus, devices, systems, kits, and an apparatus 600 according to the present teachings may include one or more windows or other viewing areas—e.g., the opening 632 described herein. For example, one or more windows may be disposed on the wrap 602 to provide visualization of catheter sites in the groin area of a patient 601, and/or in other areas. However, an apparatus 600 according to the present teachings may still maintain a relatively wide section on the patient's back that lacks such windows/openings (e.g., a quilted fabric panel may be present on the back of the apparatus 600) for comfort and stabilization during immobilization.

As shown in FIG. 6, in some aspects, feet of a patient 601 may be at least partially covered by the wrap 602 of the apparatus 600. However, in some aspects, the wrap 602 is sized and shaped (or otherwise positioned) such that feet of the patient 601 are substantially exposed when the patient 601 is swaddled by the wrap 602. This can be particularly advantageous for medical personnel or the like to access to a patient's feet for assessment of peripheral perfusion and the like.

As discussed herein, the apparatus 600 may be structurally configured to prevent the patient 601 from turning over when immobilized within the wrap 602. Also or instead, the apparatus 600 may be structurally configured to prevent the patient 601 from bending at their waist when immobilized within the wrap 602.

The patient 601 may be a post cardiac catheterization pediatric patient or the like. Other patients and user types are also or instead possible, however.

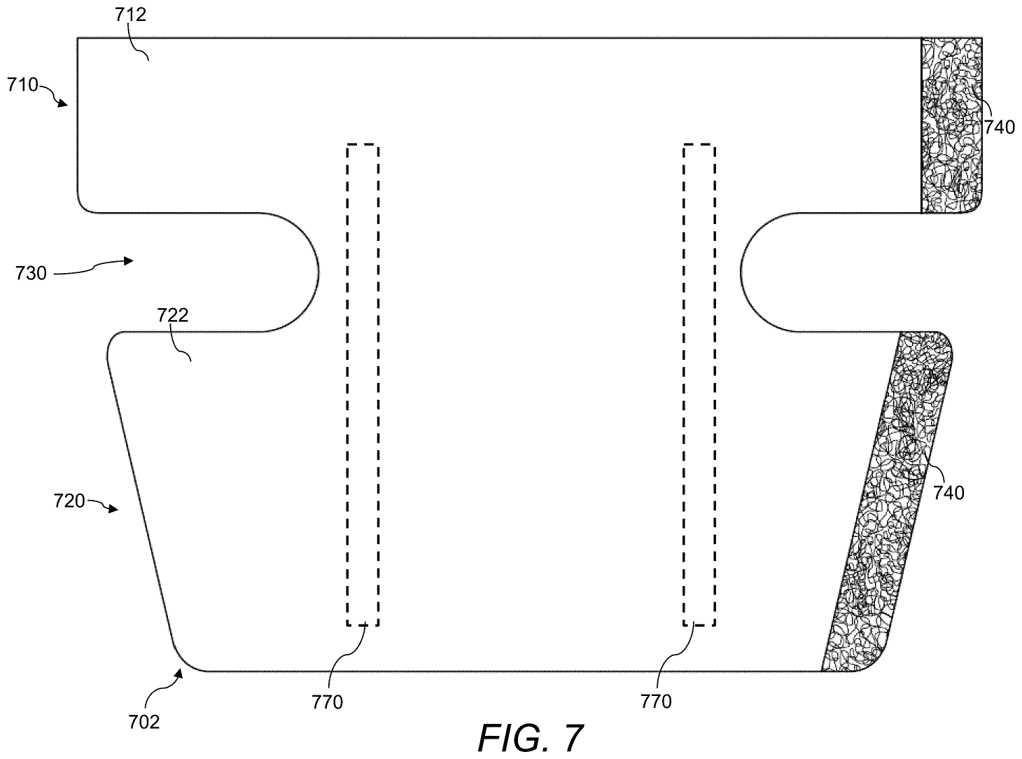

FIG. 7 is a diagram of a wrap. The wrap 702 may be the same or similar to any of the wraps described herein—e.g., including a first panel 710 having a first pair of flaps 712 and a coupler 740, a second panel 720 including a second pair of flaps 722 and a coupler 740, and a cutout 730 formed between the panels that is structurally configured to create an opening positioned axially along a patient when the wrap 702 is placed for use about the patient. The wrap 702 (and/or an apparatus, kit, or system including the wrap 702) may further include a stabilizer as described herein. Also or instead, the wrap 702 (and/or an apparatus, kit, or system including the wrap 702) may include one or more second stabilizers 770.

A second stabilizer 770 may be structurally configured to provide stability for the wrap 702 or apparatus. Also or instead, a second stabilizer 770 may be structurally configured to aid in immobilizing a patient that is swaddled by the wrap 702. In an aspect, one or more of the second stabilizers 770 may include at least one rod within the wrap 702 positioned to be disposed laterally along the patient's body when the patient is immobilized within the wrap 702. Thus, a second stabilizer 770 may be integrally contained within the wrap 702. Also or instead, a second stabilizer 770 may be coupled to the wrap 702 or another portion of an apparatus that includes the wrap 702.

Therefore, it will be understood that stabilizers described herein may include a middle liner, e.g., a foam core middle liner for the entire device that provides stability with minimal flexibility in order to decrease a risk of injury. The stabilizers may also or instead include second stabilizers 770 such as rods or the like (e.g., plastic rods) that run along lateral aspect of patient's body to provide structure and support. The rods may be disposed within rod pockets that are included on each side of the device. Ends of the rods may be covered by a material to provide protection from edges thereof.

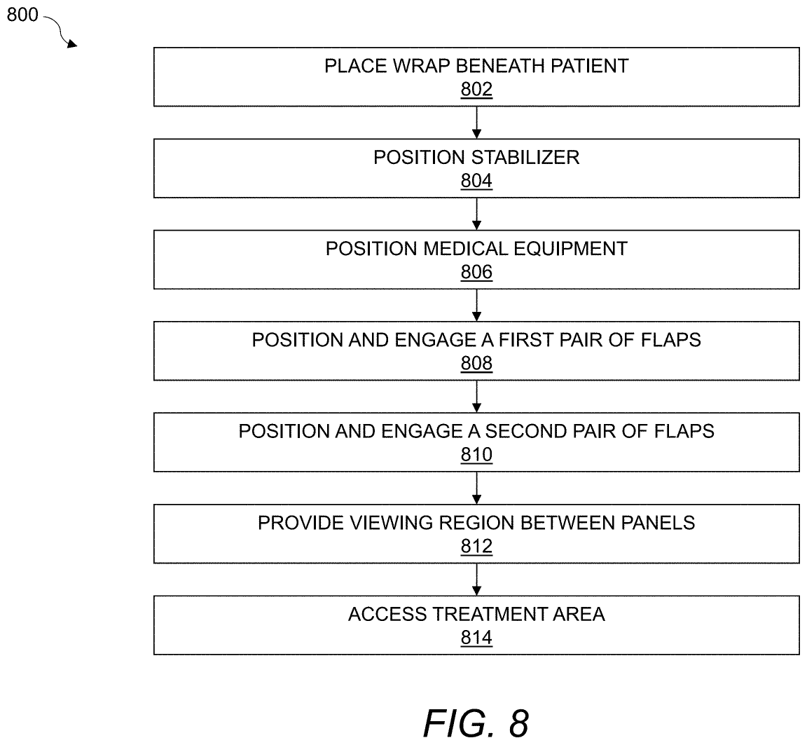

FIG. 8 is a flow chart of a method for patient immobilization. The method 800 may be performed using any of the devices, systems, kits, and apparatuses described herein, for example, with reference to FIGS. 1-7 above. Thus, the method 800 may involve the use of a device including a wrap with a window configured to permit access and/or a visual of catheter sites in the groin area (or other area of interest), and one or more stabilizers structurally configured to stabilize a patient's body when in a supine position.

As shown in step 802, the method 800 may include placing a wrap beneath a patient. The patient may be disposed in a supine position with a pair of shoulders of the patient positioned above the wrap—in this manner, after swaddling with the wrap, the patient may have substantial freedom of movement of their arms, which can provide comfort and convenience for the patient. The patient's feet may be positioned on the wrap or below the wrap. For example, in order to provide access to the patient's feet after the patient is swaddled by the wrap, a pair of feet of the patient may be positioned below the wrap. As referenced above, the wrap may be any as described herein, e.g., including a first panel having a pair of first flaps and a second panel having a pair of second flaps.

As shown in step 804, the method 800 may include positioning a stabilizer between a pair of legs of the patient, or otherwise positioning a stabilizer (e.g., positioning a stabilizer along a side of the patient or otherwise). When placed between a pair of legs of a patient, the stabilizer may displace the pair of legs from one another to impart rotational stability to the patient when immobilized within the wrap. Also or instead, a stabilizer may provide axial rigidity to resist bending at a pair of knees of the patient when immobilized within the wrap.

As shown in step 806, the method 800 may include positioning medical equipment for the patient relative to one or more of the wrap and the stabilizer. This may include, for example, running a wire, cable, IV tubing, or the like, along the patient, the wrap, and/or a stabilizer. This may also or instead include aligning a treatment site (e.g., having associated medical equipment such as a bandage, a catheter, an IV connection, and so on) in a predetermined location—e.g., alongside a cutout of the wrap.

As shown in step 808, the method 800 may include positioning a first pair of flaps of a first panel of the wrap about a torso of the patient and releasably engaging the first pair of flaps to one another about the torso. In other words, the method 800 may include swaddling a torso (or a portion thereof) of the patient with the wrap.

As shown in step 810, the method 800 may include positioning a second pair of flaps of a second panel of the wrap about the pair of legs of the patient and releasably engaging the second pair of flaps to one another about the pair of legs. In other words, the method 800 may include swaddling legs (or a portion thereof) of the patient with the wrap.

As shown in step 812, the method 800 may include providing a viewing region between panels of the wrap. This may be accomplished through the swaddling steps described above—i.e., step 808 and step 810. Thus, step 812 may include forming an opening between the first panel and the second panel through the engagement of the first pair of flaps to one another and the second pair of flaps to one another, where the opening provides a viewing region about a predetermined area of the patient (e.g., the groin of the patient) when the wrap is placed for use about the patient.

As shown in step 814, the method 800 may include accessing a treatment area of the patient through the opening. For example, this may include accessing a groin region to provide maintenance to a treatment area, and/or accessing medical equipment or the like.

Advantages of an aspect of the present teachings may include one or more of: providing mobility to a patient's arms such that they are free to move; allowing for access to a surgical site or other site(s) of interest for medical personnel without having to remove the patient from a stabilizing device (e.g., for maintenance related to dressings at a groin region, and/or for access to physiological measurement areas such as the feet); accommodating children and toddler sized patients; and so on. Leaving a patient's arms free may be advantageous because it can allow a patient to hold a computing device (e.g., a tablet or phone) or another item for distraction and/or entertainment (e.g., comfort items such as stuffed animals and the like). Also, this can allow a patient to eat or drink thereby decreasing irritability, and it can allow providers to easily see and access IVs and the like in upper extremities, and so on.

Aspects of the present teachings can be made with or without arm holes. The present teachings can be made with materials that are water resistant and/or hypoallergenic or the like. For example, materials can be washable and/or reusable, or they can be configured for one-time use (e.g., an aspect of the present teachings may include a disposable device). The present teachings may include shoulder straps, e.g., as an option if a provider deems it appropriate for a particular patient.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. An apparatus for patient immobilization, the apparatus comprising:
a wrap sized and shaped to enclose a patient in a supine position from below a pair of shoulders to above a pair of feet such that, when the wrap is placed for use about the patient, a pair of arms of the patient are free to move while a pair of legs of the patient are contained within the wrap;
a first panel of the wrap, the first panel including a first pair of flaps shaped to surround a torso of the patient, the first pair of flaps including a first coupler for releasably engaging the first pair of flaps to one another about the torso;
a second panel of the wrap, the second panel including a second pair of flaps shaped to surround a pair of legs of the patient, the second pair of flaps including a second coupler for releasably engaging the second pair of flaps to one another about the pair of legs;
an opening within the wrap, the opening positioned axially along the patient between the first panel and the second panel to provide a viewing region about a groin of the patient when the wrap is placed for use about the patient; and
a stabilizer for positioning between the pair of legs, the stabilizer displacing the pair of legs from one another to impart rotational stability to the patient when immobilized within the wrap, and the stabilizer providing axial rigidity to resist bending at a pair of knees of the patient when immobilized within the wrap.

2. The apparatus of claim 1, wherein the stabilizer is tapered.

3. The apparatus of claim 2, wherein a bottom end of the stabilizer is laterally wider than a top end of the stabilizer, and wherein the bottom end is configured for positioning substantially adjacent to ankles or feet of the patient.

4. The apparatus of claim 1, wherein the stabilizer is engaged with the wrap.

5. The apparatus of claim 4, wherein the stabilizer is stitched onto an inner lining of the wrap.

6. The apparatus of claim 1, wherein the stabilizer is removable and replaceable relative to the wrap.

7. The apparatus of claim 1, further comprising a plurality of stabilizers, wherein at least two of the plurality of stabilizers have a different size or shape.

8. The apparatus of claim 1, further comprising one or more second stabilizers, the one or more second stabilizers including at least one rod within the wrap positioned to be disposed laterally along the patient's body when the patient is immobilized within the wrap.

9. The apparatus of claim 1, wherein the viewing region is exposed to provide access to at least a portion of the patient through the viewing region when the patient is immobilized within the wrap.

10. The apparatus of claim 1, wherein the wrap is sized and shaped such that feet of the patient are exposed when the patient is swaddled by the wrap.

11. The apparatus of claim 1, wherein the apparatus is structurally configured to prevent the patient from turning over when immobilized within the wrap.

12. The apparatus of claim 1, wherein the apparatus is structurally configured to prevent the patient from bending at their waist when immobilized within the wrap.

13. The apparatus of claim 1, wherein the patient is a post cardiac catheterization pediatric patient.

14. The apparatus of claim 1, wherein the wrap is sized and shaped to immobilize patients between zero and five years of age.

15. The apparatus of claim 1, wherein at least one of the first coupler and the second coupler includes a hook and loop fastener.

16. The apparatus of claim 15, wherein the hook and loop fastener extends across an entire width of at least one flap of the first pair of flaps and the second pair of flaps.

17. The apparatus of claim 1, wherein the wrap includes one or more elastic sections configured to conform to a shape of the patient.

18. A method for patient immobilization, the method comprising:
placing a wrap beneath a patient in a supine position with a pair of shoulders of the patient positioned above the wrap and a pair of feet of the patient positioned below the wrap, the wrap including a first panel having a pair of first flaps and a second panel having a pair of second flaps;
positioning a stabilizer between a pair of legs of the patient, the stabilizer displacing the pair of legs from one another to impart rotational stability to the patient when immobilized within the wrap, and the stabilizer providing axial rigidity to resist bending at a pair of knees of the patient when immobilized within the wrap;
positioning the first pair of flaps of the first panel about a torso of the patient and releasably engaging the first pair of flaps to one another about the torso;
positioning the second pair of flaps of the second panel about the pair of legs of the patient and releasably engaging the second pair of flaps to one another about the pair of legs; and
forming an opening between the first panel and the second panel through the engagement of the first pair of flaps to one another and the second pair of flaps to one another, the opening providing a viewing region about a groin of the patient when the wrap is placed for use about the patient.

19. The method of claim 18, further comprising accessing a treatment area of the patient through the opening.

20. The method of claim 18, further comprising positioning medical equipment relative to the wrap before engaging flaps of the wrap.
